(19) United States
(12) Patent Application Publication
(10) Pub. No.: US 2026/0237469 A1
(43) Pub. Date: Aug. 13, 2026

(54) SYSTEM FOR MOLECULAR FISHING USING BAIT-COMPOUNDS FOR SEMI-TARGETED SEARCH WITHIN MOLECULAR NETWORKS

(71) Applicant: THERMO FISHER SCIENTIFIC (BREMEN) GMBH, Bremen (DE)

(72) Inventor: Martin Strohalm, Bremen (DE)

(21) Appl. No.: 19/155,361

(22) PCT Filed: Aug. 29, 2024
(86) PCT No.: PCT/EP2024/054317

§ 371 Date: Aug. 11, 2025

Related U.S. Application Data
(60) Provisional application No. 63/486,010, filed on Feb. 20, 2023.

(51) Int. Cl. G16C 20/20; G16C 20/60; G16C 20/80; H01J 49/00

(52) U.S. Cl. CPC G16C 20/20; G16C 20/60; G16C 20/80; H01J 49/0036

(57) ABSTRACT

Disclosed herein are scientific instrument support systems, as well as related methods, computing devices, and computer-readable media. For example, in some embodiments, a mass spectrometry instrument support apparatus may include: first logic to receive fragmentation data of a reference compound; second logic to provide the fragmentation data to a molecular networking algorithm as a virtual bait compound; third logic to annotate the reference compound and generate a cluster of compounds within a sample and surrounding the annotated reference compound; and fourth logic to provide for display an overview of structural relationships between the cluster of compounds.

### RELATED APPLICATION

This application claims the benefit of U.S. Provisional Application No. 63/486,010, filed Feb. 20, 2024, the entire content of which is hereby incorporated by reference herein

### BACKGROUND

Mass spectrometry is a technique for detecting, identifying, and quantifying molecules within samples based on their molecular mass-to-charge ratio after ionization. Data generated by mass spectrometry operations may be used in proteomics and other scientific applications.

### BRIEF DESCRIPTION OF THE DRAWINGS

Embodiments will be readily understood by the following detailed description in conjunction with the accompanying drawings. To facilitate this description, like reference numerals designate like structural elements. Embodiments are illustrated by way of example, not by way of limitation, in the figures of the accompanying drawings.

FIG. 1 is a block diagram of an example mass spectrometry instrument support module, in accordance with various embodiments.

FIG. 2 is a flow diagram of an example method for mass spectrometry data analysis, in accordance with various embodiments.

FIG. 3 depicts an example output showing clusters of compounds within a sample and surrounding an annotated reference compound.

FIG. 4 is an example of a graphical user interface that may be used in the performance of some or all of the support methods disclosed herein, in accordance with various embodiments.

FIG. 4 is a block diagram of an example computing device that may perform some or all of the mass spectrometry methods disclosed herein, in accordance with various embodiments.

FIG. 5 is a block diagram of an example scientific instrument support system in which some or all of the methods disclosed herein may be performed, in accordance with various embodiments.

### DETAILED DESCRIPTION

Disclosed herein are scientific instrument support systems, as well as related methods, computing devices, and computer-readable media. For example, in some embodiments, a mass spectrometry instrument support apparatus may include: first logic to receive fragmentation data of a reference compound; second logic to provide the fragmentation data to molecular networking algorithm as a virtual bait compound; third logic to annotate the reference compound and generate a cluster of compounds within a sample and surrounding the annotated reference compound; and fourth logic to provide for display an overview of structural relationships between the cluster of compounds.

The scientific instrument support embodiments disclosed herein may achieve improved performance relative to conventional approaches. As discussed in further detail below, in many experiment designs, scientists are searching for unknown compounds structurally related to a known parent or reference substance to submit them for further analysis. Finding such candidate compounds within complex samples can be time consuming and complicated task even for experienced scientists.

The embodiments disclosed herein may include systems (e.g., tools) to provide visual overview of structural relationships between compounds detected by mass spectrometry. By this technique, compounds are clustered together using similarity scoring between their fragmentation mass spectra. Although these systems provide significant help in sample overview, compound identification and validation, there are scenarios, where finding candidate compounds remains intricate.

Accordingly, in some embodiments, the described system includes features to facilitate a semi-targeted search for clusters of structurally related compounds and improve mass spectrometric analysis of small molecules such as metabolic pathway analysis, degradation product analysis, forensic analysis, and the like. In common cases, a cluster of related compounds can be located, even within complex molecular network, if any of its members is identified and annotated. The search can be performed directly by subpart of its name or by observing the structures. There are important cases, however, where a sample contains just unknown derivatives of a reference compound and where corresponding cluster becomes virtually invisible. As an example, for a metabolomic study of a newly designed drug, a sample may include metabolites that cannot be directly identified due to lack of reference spectra.

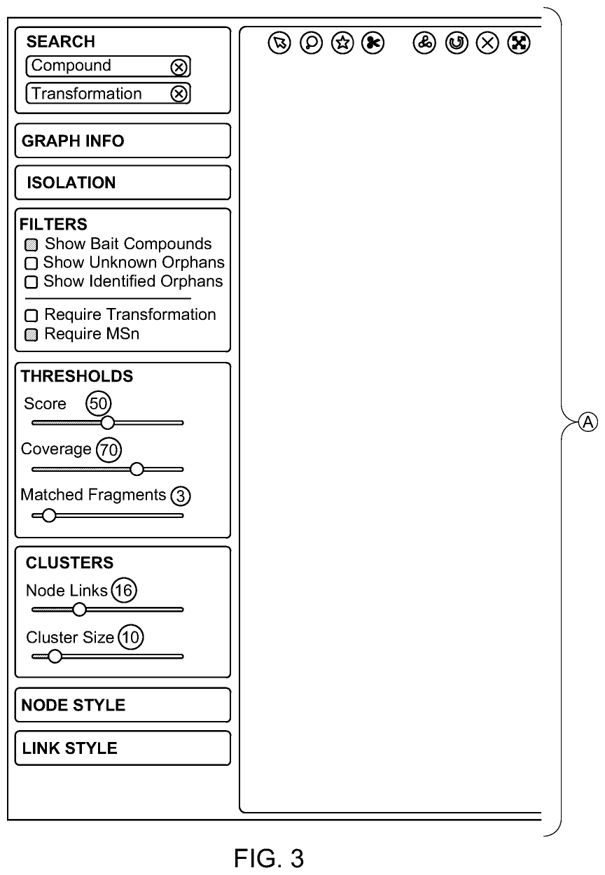

To address the above-mentioned problem, in some embodiments, the described system receives fragmentation data of a clean reference compound. The substance is then injected directly into a sample, which are measured together in the same run. In such examples, the cluster is created around an annotated reference compound, which makes it easy to find (see FIG. 3). In some cases, however, the reference compound cannot be injected into a sample and thus measured directly with other compounds. Fortunately, in such cases, an in-silico technique can be employed where user-defined fragmentation data of a reference compound can be injected into a molecular networking algorithm as a virtual “bait compound.” This allows users to perform semi-targeted search for potentially interesting clusters. Moreover, the source of fragmentation data of a reference compound is not limited to a real acquired tandem mass spectrometry (MS2) spectrum of that compound. Instead, the source may be, for example, an artificially predicted spectrum based on known structure, a cumulative spectrum based on diagnostic fragments of a class of similar compounds, or the like.

In some cases, the bait compounds are practically indistinguishable from the real features detected in the sample. Accordingly, in some embodiments, fragmentation data of each bait compound is compared with each detected feature and a similarity between them is determined (e.g., in the same way as for normal molecular network). Moreover, this similarity may be tuned depending on the type of bait compound or fragmentation data origin. For instance, in-silico generated fragments typically include many false positive masses, causing a lowed similarity score. For compound classes, no precursor mass is available and therefore only direct fragment matches can be used, causing a lowed similarity the score as well.

In some embodiments, the system incorporates tools to seamlessly incorporate the bait compounds making them prominent and easy to locate. For example, bait compounds within an “isolation mode” may be provided, where users can limit the displayed graph depth and release the scoring restrictions without losing control over the displayed graph. This allows direct analysis of only the compounds related to selected bait compound. Moreover, the described system allows user to insert custom fragmentation data of compounds or compound classes into molecular network and mark clusters of structurally related compounds for easy access and further analysis. Additionally, the semi-targeted search functionality reduces the effort needed to manually locate structurally related compounds of potential interest. By artificially inserting user-defined fragmentation data of individual compounds or compound classes, clusters of structurally related compounds are marked and easy to locate even within very complex samples. This strategy provides invaluable help in discovery of compounds derived from new substances, where such compounds are unknown to identification libraries.

In the following detailed description, reference is made to the accompanying drawings that form a part hereof wherein like numerals designate like parts throughout, and in which is shown, by way of illustration, embodiments that may be practiced. It is to be understood that other embodiments may be utilized, and structural or logical changes may be made, without departing from the scope of the present disclosure. Therefore, the following detailed description is not to be taken in a limiting sense.

Various operations may be described as multiple discrete actions or operations in turn, in a manner that is most helpful in understanding the subject matter disclosed herein. However, the order of description should not be construed as to imply that these operations are necessarily order dependent. In particular, these operations may not be performed in the order of presentation. Operations described may be performed in a different order from the described embodiment. Various additional operations may be performed, and/or described operations may be omitted in additional embodiments.

For the purposes of the present disclosure, the phrases “A and/or B” and “A or B” mean (A), (B), or (A and B). For the purposes of the present disclosure, the phrases “A, B, and/or C” and “A, B, or C” mean (A), (B), (C), (A and B), (A and C), (B and C), or (A, B, and C). Although some elements may be referred to in the singular (e.g., “a processing device”), any appropriate elements may be represented by multiple instances of that element, and vice versa. For example, a set of operations described as performed by a processing device may be implemented with different ones of the operations performed by different processing devices.

The description uses the phrases “an embodiment,” “various embodiments,” and “some embodiments,” each of which may refer to one or more of the same or different embodiments. Furthermore, the terms “comprising,” “including,” “having,” and the like, as used with respect to embodiments of the present disclosure, are synonymous. When used to describe a range of dimensions, the phrase “between X and Y” represents a range that includes X and Y. As used herein, an “apparatus” may refer to any individual device, collection of devices, part of a device, or collections of parts of devices. The drawings are not necessarily to scale.

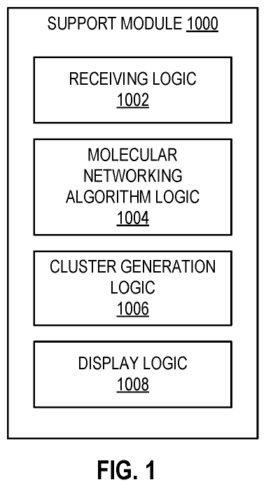

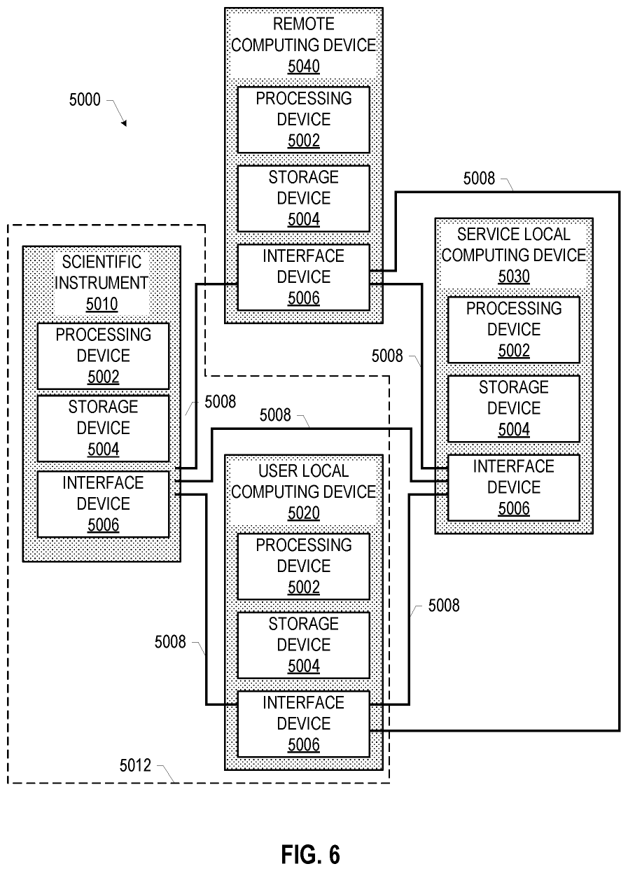

FIG. 1 is a block diagram of a mass spectrometry support module 1000 for providing a visual overview of structural relationships between compounds detected by mass spectrometry, in accordance with various embodiments. The support module 1000 may be implemented by circuitry (e.g., including electrical and/or optical components), such as a programmed computing device. The logic of the support module 1000 may be included in a single computing device or may be distributed across multiple computing devices that are in communication with each other as appropriate. Examples of computing devices that may, singly or in combination, implement the support module 1000 are discussed herein with reference to the computing device 4000 of FIG. 5, and examples of systems of interconnected computing devices, in which the support module 1000 may be implemented across one or more of the computing devices, is discussed herein with reference to the support system 5000 of FIG. 6.

The support module 1000 may include receiving logic 1002, molecular networking algorithm logic 1004, cluster generation logic 1006, and display logic 1008. As used herein, the term “logic” may include an apparatus that is to perform a set of operations associated with the logic. For example, any of the logic elements included in the support module 1000 may be implemented by one or more computing devices programmed with instructions to cause one or more processing devices of the computing devices to perform the associated set of operations. In a particular embodiment, a logic element may include one or more non-transitory computer-readable media having instructions thereon that, when executed by one or more processing devices of one or more computing devices, cause the one or more computing devices to perform the associated set of operations. As used herein, the term “module” may refer to a collection of one or more logic elements that, together, perform one or more functions associated with the module. Different ones of the logic elements in a module may take the same form or may take different forms. For example, some logic in a module may be implemented by a programmed general-purpose processing device, while other logic in a module may be implemented by an application-specific integrated circuit (ASIC). In another example, different ones of the logic elements in a module may be associated with different sets of instructions executed by one or more processing devices. A module may not include all of the logic elements depicted in the associated drawing; for example, a module may include a subset of the logic elements depicted in the associated drawing when that module is to perform a subset of the operations discussed herein with reference to that module.

The receiving logic 1002 may be configured to receive fragmentation data of a reference compound. In some embodiments, a source of the fragmentation data of the reference compound is an artificially predicted spectrum based on known structure, a cumulative spectrum based on diagnostic fragments of a class of similar compounds, or the like. In some embodiments, the fragmentation data of a reference compound is user-defined.

The molecular networking algorithm logic 1004 may be configured provide the fragmentation data to molecular networking algorithm as a virtual bait compound.

The cluster generation logic 1006 may be configured to annotate the reference compound and generate a cluster of compounds within a sample and surrounding the annotated reference compound. In some embodiments, the compounds are clustered by determining a similarity between the fragmentation data of the reference compound and real features detected in the sample. In some embodiments, determining the similarity between the fragmentation data and the real features is tuned depending on the type of the virtual bait compound or an origin of the fragmentation data.

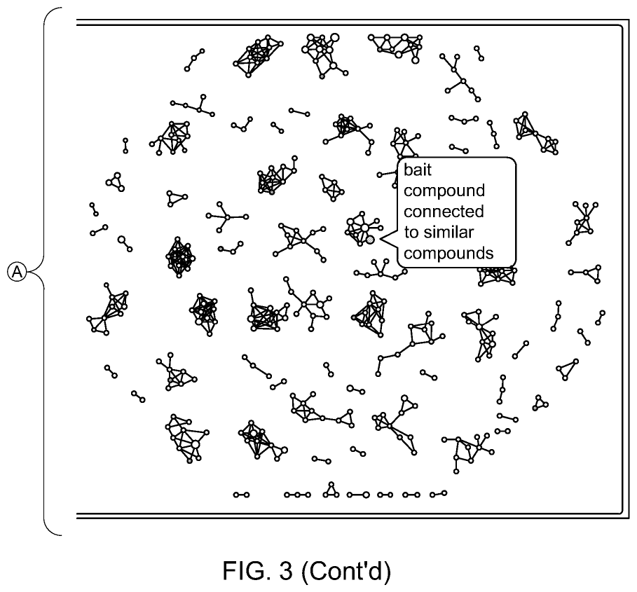

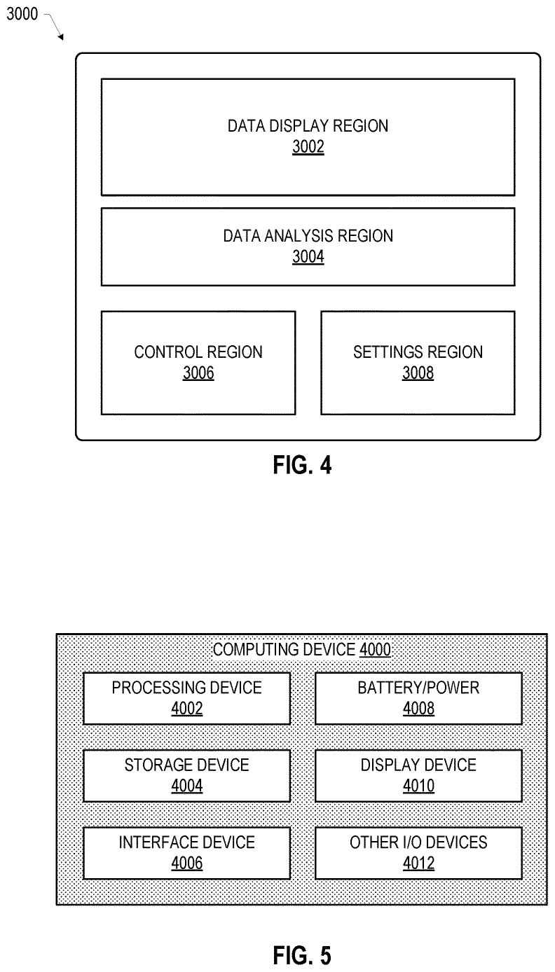

The display logic 1008 may provide to a user, through a GUI (such as the GUI 3000 of FIG. 4), an overview of structural relationships between the cluster of compounds made available by the cluster generation logic 1006. In some embodiments, the overview allows users to perform semi-targeted search for potentially interesting clusters. In some embodiments, the overview incorporates the virtual bait compound within an isolation mode where a user can limit a depth of a displayed graph depth and release scoring restrictions without losing control over the graph.

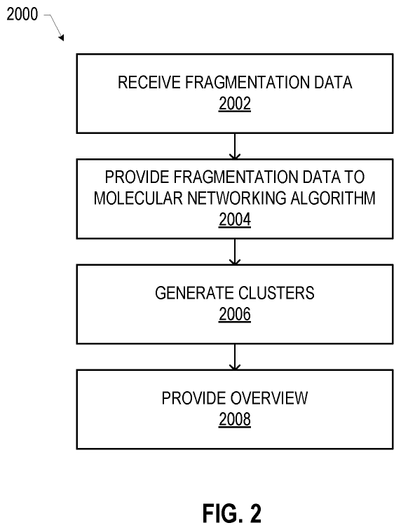

FIG. 2 is a flow diagram of a method 2000 of performing support operations, in accordance with various embodiments. Although the operations of the method 2000 may be illustrated with reference to particular embodiments disclosed herein (e.g., the scientific instrument support modules 1000 discussed herein with reference to FIG. 1, the GUI 3000 discussed herein with reference to FIG. 4, the computing devices 4000 discussed herein with reference to FIG. 5, and/or the scientific instrument support system 5000 discussed herein with reference to FIG. 6), the method 2000 may be used in any suitable setting to perform any suitable support operations. Operations are illustrated once each and in a particular order in FIG. 2, but the operations may be reordered and/or repeated as desired and appropriate (e.g., different operations performed may be performed in parallel, as suitable).

At 2002, first operations may be performed. For example, the receiving logic 1002 of the support module 1000 may perform the operations of 2002. The first operations may include receiving fragmentation data of a reference compound.

At 2004, second operations may be performed. For example, the molecular networking algorithm logic 1004 of the support module 1000 may perform the operations of 2004. The second operations may include providing the fragmentation data to molecular networking algorithm as a virtual bait compound.

At 2006, third operations may be performed. For example, the cluster generation logic 1006 of the support module 1000 may perform the operations of 2006. The third operations may include annotating the reference compound and generating a cluster of compounds within a sample and surrounding the annotated reference compound.

At 2008, fourth operations may be performed. For example, the display logic 1008 of the support module 1000 may perform the operations of 2008. The fourth operations may include displaying an overview of structural relationships between the cluster of compounds.

FIG. 3 depicts an example output showing clusters of compounds within a sample and surrounding an annotated reference compound.

The scientific instrument support methods disclosed herein may include interactions with a human user (e.g., via the user local computing device 5020 discussed herein with reference to FIG. 6). These interactions may include providing information to the user (e.g., information regarding the operation of a scientific instrument such as the scientific instrument 5010 of FIG. 6, information regarding a sample being analyzed or other test or measurement performed by a scientific instrument, information retrieved from a local or remote database, or other information) or providing an option for a user to input commands (e.g., to control the operation of a scientific instrument such as the scientific instrument 5010 of FIG. 6, or to control the analysis of data generated by a scientific instrument), queries (e.g., to a local or remote database), or other information. In some embodiments, these interactions may be performed through a graphical user interface (GUI) that includes a visual display on a display device (e.g., the display device 4010 discussed herein with reference to FIG. 4) that provides outputs to the user and/or prompts the user to provide inputs (e.g., via one or more input devices, such as a keyboard, mouse, trackpad, or touchscreen, included in the other I/O devices 4012 discussed herein with reference to FIG. 4). The scientific instrument support systems disclosed herein may include any suitable GUIs for interaction with a user.

FIG. 4 depicts an example GUI 3000 that may be used in the performance of some or all of the support methods disclosed herein, in accordance with various embodiments. As noted above, the GUI 3000 may be provided on a display device (e.g., the display device 4010 discussed herein with reference to FIG. 5) of a computing device (e.g., the computing device 4000 discussed herein with reference to FIG. 5) of a scientific instrument support system (e.g., the scientific instrument support system 5000 discussed herein with reference to FIG. 6), and a user may interact with the GUI 3000 using any suitable input device (e.g., any of the input devices included in the other I/O devices 4012 discussed herein with reference to FIG. 5) and input technique (e.g., movement of a cursor, motion capture, facial recognition, gesture detection, voice recognition, actuation of buttons, etc.).

The GUI 3000 may include a data display region 3002, a data analysis region 3004, a scientific instrument control region 3006, and a settings region 3008. The particular number and arrangement of regions depicted in FIG. 4 is simply illustrative, and any number and arrangement of regions, including any desired features, may be included in a GUI 3000.

The data display region 3002 may display data generated by a scientific instrument (e.g., the scientific instrument 5010 discussed herein with reference to FIG. 6). For example, the data display region 3002 may provide to a user an option to provide fragmentation data of a reference compound and an overview of structural relationships between the cluster of compounds.

The data analysis region 3004 may display the results of data analysis. For example, the data analysis region 3004 may display an overview of structural relationships between the cluster of compounds. In some embodiments, the data display region 3002 and the data analysis region 3004 may be combined in the GUI 3000 (e.g., to include data output from a scientific instrument, and some analysis of the data, in a common graph or region).

The scientific instrument control region 3006 may include options that allow the user to control a scientific instrument (e.g., the scientific instrument 5010 discussed herein with reference to FIG. 6). The settings region 3008 may include options that allow the user to control the features and functions of the GUI 3000 (and/or other GUIs) and/or perform common computing operations with respect to the data display region 3002 and data analysis region 3004 (e.g., saving data on a storage device, such as the storage device 4004 discussed herein with reference to FIG. 5, sending data to another user, labeling data, etc.).

As noted above, the scientific instrument support module 1000 may be implemented by one or more computing devices. FIG. 5 is a block diagram of a computing device 4000 that may perform some or all of the scientific instrument support methods disclosed herein, in accordance with various embodiments. In some embodiments, the scientific instrument support module 1000 may be implemented by a single computing device 4000 or by multiple computing devices 4000. Further, as discussed below, a computing device 4000 (or multiple computing devices 4000) that implements the scientific instrument support module 1000 may be part of one or more of the scientific instrument 5010, the user local computing device 5020, the service local computing device 5030, or the remote computing device 5040 of FIG. 6.

The computing device 4000 of FIG. 5 is illustrated as having a number of components, but any one or more of these components may be omitted or duplicated, as suitable for the application and setting. In some embodiments, some or all of the components included in the computing device 4000 may be attached to one or more motherboards and enclosed in a housing (e.g., including plastic, metal, and/or other materials). In some embodiments, some these components may be fabricated onto a single system-on-a-chip (SoC) (e.g., an SoC may include one or more processing devices 4002 and one or more storage devices 4004). Additionally, in various embodiments, the computing device 4000 may not include one or more of the components illustrated in FIG. 5, but may include interface circuitry (not shown) for coupling to the one or more components using any suitable interface (e.g., a Universal Serial Bus (USB) interface, a High-Definition Multimedia Interface (HDMI) interface, a Controller Area Network (CAN) interface, a Serial Peripheral Interface (SPI) interface, an Ethernet interface, a wireless interface, or any other appropriate interface). For example, the computing device 4000 may not include a display device 4010, but may include display device interface circuitry (e.g., a connector and driver circuitry) to which a display device 4010 may be coupled.

The computing device 4000 may include a processing device 4002 (e.g., one or more processing devices). As used herein, the term “processing device” may refer to any device or portion of a device that processes electronic data from registers and/or memory to transform that electronic data into other electronic data that may be stored in registers and/or memory. The processing device 4002 may include one or more digital signal processors (DSPs), application-specific integrated circuits (ASICs), central processing units (CPUs), graphics processing units (GPUs), cryptoprocessors (specialized processors that execute cryptographic algorithms within hardware), server processors, or any other suitable processing devices.

The computing device 4000 may include a storage device 4004 (e.g., one or more storage devices). The storage device 4004 may include one or more memory devices such as random access memory (RAM) (e.g., static RAM (SRAM) devices, magnetic RAM (MRAM) devices, dynamic RAM (DRAM) devices, resistive RAM (RRAM) devices, or conductive-bridging RAM (CBRAM) devices), hard drive-based memory devices, solid-state memory devices, networked drives, cloud drives, or any combination of memory devices. In some embodiments, the storage device 4004 may include memory that shares a die with a processing device 4002. In such an embodiment, the memory may be used as cache memory and may include embedded dynamic random access memory (eDRAM) or spin transfer torque magnetic random access memory (STT-MRAM), for example. In some embodiments, the storage device 4004 may include non-transitory computer readable media having instructions thereon that, when executed by one or more processing devices (e.g., the processing device 4002), cause the computing device 4000 to perform any appropriate ones of or portions of the methods disclosed herein.

The computing device 4000 may include an interface device 4006 (e.g., one or more interface devices 4006). The interface device 4006 may include one or more communication chips, connectors, and/or other hardware and software to govern communications between the computing device 4000 and other computing devices. For example, the interface device 4006 may include circuitry for managing wireless communications for the transfer of data to and from the computing device 4000. The term “wireless” and its derivatives may be used to describe circuits, devices, systems, methods, techniques, communications channels, etc., that may communicate data through the use of modulated electromagnetic radiation through a nonsolid medium. The term does not imply that the associated devices do not contain any wires, although in some embodiments they might not. Circuitry included in the interface device 4006 for managing wireless communications may implement any of a number of wireless standards or protocols, including but not limited to Institute for Electrical and Electronic Engineers (IEEE) standards including Wi-Fi (IEEE 802.11 family), IEEE 802.16 standards (e.g., IEEE 802.16-2005 Amendment), Long-Term Evolution (LTE) project along with any amendments, updates, and/or revisions (e.g., advanced LTE project, ultra mobile broadband (UMB) project (also referred to as “3GPP2”), etc.). In some embodiments, circuitry included in the interface device 4006 for managing wireless communications may operate in accordance with a Global System for Mobile Communication (GSM), General Packet Radio Service (GPRS), Universal Mobile Telecommunications System (UMTS), High Speed Packet Access (HSPA), Evolved HSPA (E-HSPA), or LTE network. In some embodiments, circuitry included in the interface device 4006 for managing wireless communications may operate in accordance with Enhanced Data for GSM Evolution (EDGE), GSM EDGE Radio Access Network (GERAN), Universal Terrestrial Radio Access Network (UTRAN), or Evolved UTRAN (E-UTRAN). In some embodiments, circuitry included in the interface device 4006 for managing wireless communications may operate in accordance with Code Division Multiple Access (CDMA), Time Division Multiple Access (TDMA), Digital Enhanced Cordless Telecommunications (DECT), Evolution-Data Optimized (EV-DO), and derivatives thereof, as well as any other wireless protocols that are designated as 3G, 4G, 5G, and beyond. In some embodiments, the interface device 4006 may include one or more antennas (e.g., one or more antenna arrays) to receipt and/or transmission of wireless communications.

In some embodiments, the interface device 4006 may include circuitry for managing wired communications, such as electrical, optical, or any other suitable communication protocols. For example, the interface device 4006 may include circuitry to support communications in accordance with Ethernet technologies. In some embodiments, the interface device 4006 may support both wireless and wired communication, and/or may support multiple wired communication protocols and/or multiple wireless communication protocols. For example, a first set of circuitry of the interface device 4006 may be dedicated to shorter-range wireless communications such as Wi-Fi or Bluetooth, and a second set of circuitry of the interface device 4006 may be dedicated to longer-range wireless communications such as global positioning system (GPS), EDGE, GPRS, CDMA, WiMAX, LTE, EV-DO, or others. In some embodiments, a first set of circuitry of the interface device 4006 may be dedicated to wireless communications, and a second set of circuitry of the interface device 4006 may be dedicated to wired communications.

The computing device 4000 may include battery/power circuitry 4008. The battery/power circuitry 4008 may include one or more energy storage devices (e.g., batteries or capacitors) and/or circuitry for coupling components of the computing device 4000 to an energy source separate from the computing device 4000 (e.g., AC line power).

The computing device 4000 may include a display device 4010 (e.g., multiple display devices). The display device 4010 may include any visual indicators, such as a heads-up display, a computer monitor, a projector, a touchscreen display, a liquid crystal display (LCD), a light-emitting diode display, or a flat panel display.

The computing device 4000 may include other input/output (I/O) devices 4012. The other I/O devices 4012 may include one or more audio output devices (e.g., speakers, headsets, earbuds, alarms, etc.), one or more audio input devices (e.g., microphones or microphone arrays), location devices (e.g., GPS devices in communication with a satellite-based system to receive a location of the computing device 4000, as known in the art), audio codecs, video codecs, printers, sensors (e.g., thermocouples or other temperature sensors, humidity sensors, pressure sensors, vibration sensors, accelerometers, gyroscopes, etc.), image capture devices such as cameras, keyboards, cursor control devices such as a mouse, a stylus, a trackball, or a touchpad, bar code readers, Quick Response (QR) code readers, or radio frequency identification (RFID) readers, for example.

The computing device 4000 may have any suitable form factor for its application and setting, such as a handheld or mobile computing device (e.g., a cell phone, a smart phone, a mobile internet device, a tablet computer, a laptop computer, a netbook computer, an ultrabook computer, a personal digital assistant (PDA), an ultra mobile personal computer, etc.), a desktop computing device, or a server computing device or other networked computing component.

One or more computing devices implementing any of the scientific instrument support modules or methods disclosed herein may be part of a scientific instrument support system. FIG. 6 is a block diagram of an example scientific instrument support system 5000 in which some or all of the scientific instrument support methods disclosed herein may be performed, in accordance with various embodiments. The scientific instrument support modules and methods disclosed herein (e.g., the scientific instrument support module 1000 of FIG. 1 and the method 2000 of FIG. 2) may be implemented by one or more of the scientific instrument 5010, the user local computing device 5020, the service local computing device 5030, or the remote computing device 5040 of the scientific instrument support system 5000.

Any of the scientific instrument 5010, the user local computing device 5020, the service local computing device 5030, or the remote computing device 5040 may include any of the embodiments of the computing device 4000 discussed herein with reference to FIG. 5, and any of the scientific instrument 5010, the user local computing device 5020, the service local computing device 5030, or the remote computing device 5040 may take the form of any appropriate ones of the embodiments of the computing device 4000 discussed herein with reference to FIG. 5.

The scientific instrument 5010, the user local computing device 5020, the service local computing device 5030, or the remote computing device 5040 may each include a processing device 5002, a storage device 5004, and an interface device 5006. The processing device 5002 may take any suitable form, including the form of any of the processing devices 4002 discussed herein with reference to FIG. 4, and the processing devices 5002 included in different ones of the scientific instrument 5010, the user local computing device 5020, the service local computing device 5030, or the remote computing device 5040 may take the same form or different forms. The storage device 5004 may take any suitable form, including the form of any of the storage devices 5004 discussed herein with reference to FIG. 4, and the storage devices 5004 included in different ones of the scientific instrument 5010, the user local computing device 5020, the service local computing device 5030, or the remote computing device 5040 may take the same form or different forms. The interface device 5006 may take any suitable form, including the form of any of the interface devices 4006 discussed herein with reference to FIG. 4, and the interface devices 5006 included in different ones of the scientific instrument 5010, the user local computing device 5020, the service local computing device 5030, or the remote computing device 5040 may take the same form or different forms.

The scientific instrument 5010, the user local computing device 5020, the service local computing device 5030, and the remote computing device 5040 may be in communication with other elements of the scientific instrument support system 5000 via communication pathways 5008. The communication pathways 5008 may communicatively couple the interface devices 5006 of different ones of the elements of the scientific instrument support system 5000, as shown, and may be wired or wireless communication pathways (e.g., in accordance with any of the communication techniques discussed herein with reference to the interface devices 4006 of the computing device 4000 of FIG. 5). The particular scientific instrument support system 5000 depicted in FIG. 6 includes communication pathways between each pair of the scientific instrument 5010, the user local computing device 5020, the service local computing device 5030, and the remote computing device 5040, but this “fully connected” implementation is simply illustrative, and in various embodiments, various ones of the communication pathways 5008 may be absent. For example, in some embodiments, a service local computing device 5030 may not have a direct communication pathway 5008 between its interface device 5006 and the interface device 5006 of the scientific instrument 5010, but may instead communicate with the scientific instrument 5010 via the communication pathway 5008 between the service local computing device 5030 and the user local computing device 5020 and the communication pathway 5008 between the user local computing device 5020 and the scientific instrument 5010.

The user local computing device 5020 may be a computing device (e.g., in accordance with any of the embodiments of the computing device 4000 discussed herein) that is local to a user of the scientific instrument 5010. In some embodiments, the user local computing device 5020 may also be local to the scientific instrument 5010, but this need not be the case; for example, a user local computing device 5020 that is in a user's home or office may be remote from, but in communication with, the scientific instrument 5010 so that the user may use the user local computing device 5020 to control and/or access data from the scientific instrument 5010. In some embodiments, the user local computing device 5020 may be a laptop, smartphone, or tablet device. In some embodiments the user local computing device 5020 may be a portable computing device.

The service local computing device 5030 may be a computing device (e.g., in accordance with any of the embodiments of the computing device 4000 discussed herein) that is local to an entity that services the scientific instrument 5010. For example, the service local computing device 5030 may be local to a manufacturer of the scientific instrument 5010 or to a third-party service company. In some embodiments, the service local computing device 5030 may communicate with the scientific instrument 5010, the user local computing device 5020, and/or the remote computing device 5040 (e.g., via a direct communication pathway 5008 or via multiple “indirect” communication pathways 5008, as discussed above) to receive data regarding the operation of the scientific instrument 5010, the user local computing device 5020, and/or the remote computing device 5040 (e.g., the results of self-tests of the scientific instrument 5010, calibration coefficients used by the scientific instrument 5010, the measurements of sensors associated with the scientific instrument 5010, etc.). In some embodiments, the service local computing device 5030 may communicate with the scientific instrument 5010, the user local computing device 5020, and/or the remote computing device 5040 (e.g., via a direct communication pathway 5008 or via multiple “indirect” communication pathways 5008, as discussed above) to transmit data to the scientific instrument 5010, the user local computing device 5020, and/or the remote computing device 5040 (e.g., to update programmed instructions, such as firmware, in the scientific instrument 5010, to initiate the performance of test or calibration sequences in the scientific instrument 5010, to update programmed instructions, such as software, in the user local computing device 5020 or the remote computing device 5040, etc.). A user of the scientific instrument 5010 may utilize the scientific instrument 5010 or the user local computing device 5020 to communicate with the service local computing device 5030 to report a problem with the scientific instrument 5010 or the user local computing device 5020, to request a visit from a technician to improve the operation of the scientific instrument 5010, to order consumables or replacement parts associated with the scientific instrument 5010, or for other purposes.

The remote computing device 5040 may be a computing device (e.g., in accordance with any of the embodiments of the computing device 4000 discussed herein) that is remote from the scientific instrument 5010 and/or from the user local computing device 5020. In some embodiments, the remote computing device 5040 may be included in a datacenter or other large-scale server environment. In some embodiments, the remote computing device 5040 may include network-attached storage (e.g., as part of the storage device 5004). The remote computing device 5040 may store data generated by the scientific instrument 5010, perform analyses of the data generated by the scientific instrument 5010 (e.g., in accordance with programmed instructions), facilitate communication between the user local computing device 5020 and the scientific instrument 5010, and/or facilitate communication between the service local computing device 5030 and the scientific instrument 5010.

In some embodiments, one or more of the elements of the scientific instrument support system 5000 illustrated in FIG. 6 may not be present. Further, in some embodiments, multiple ones of various ones of the elements of the scientific instrument support system 5000 of FIG. 6 may be present. For example, a scientific instrument support system 5000 may include multiple user local computing devices 5020 (e.g., different user local computing devices 5020 associated with different users or in different locations). In another example, a scientific instrument support system 5000 may include multiple scientific instruments 5010, all in communication with service local computing device 5030 and/or a remote computing device 5040; in such an embodiment, the service local computing device 5030 may monitor these multiple scientific instruments 5010, and the service local computing device 5030 may cause updates or other information may be “broadcast” to multiple scientific instruments 5010 at the same time. Different ones of the scientific instruments 5010 in a scientific instrument support system 5000 may be located close to one another (e.g., in the same room) or farther from one another (e.g., on different floors of a building, in different buildings, in different cities, etc.). In some embodiments, a scientific instrument 5010 may be connected to an Internet-of-Things (IoT) stack that allows for command and control of the scientific instrument 5010 through a web-based application, a virtual or augmented reality application, a mobile application, and/or a desktop application. Any of these applications may be accessed by a user operating the user local computing device 5020 in communication with the scientific instrument 5010 by the intervening remote computing device 5040. In some embodiments, a scientific instrument 5010 may be sold by the manufacturer along with one or more associated user local computing devices 5020 as part of a local scientific instrument computing unit 5012.

The following paragraphs provide various examples of the embodiments disclosed herein.

Example 1 is a mass spectrometry support apparatus including first logic to receive fragmentation data of a reference compound; second logic to provide the fragmentation data to molecular networking algorithm as a virtual bait compound; third logic to annotate the reference compound and generate a cluster of compounds within a sample and surrounding the annotated reference compound; and fourth logic to provide for display an overview of structural relationships between the cluster of compounds

Example 2 includes the subject matter of Example 1, and further specifies that the compounds are clustered by determining a similarity between the fragmentation data of the reference compound and real features detected in the sample.

Example 3 includes the subject matter of any of Examples 1 and 2, and further specifies that determining the similarity between the fragmentation data and the real features is tuned depending on the type of the virtual bait compound or an origin of the fragmentation data.

Example 4 includes the subject matter of any of Examples 1-3, and further specifies that a source of the fragmentation data of the reference compound comprises an artificially predicted spectrum based on known structure or a cumulative spectrum based on diagnostic fragments of a class of similar compounds.

Example 5 includes the subject matter of any of Examples 1-4, and further specifies that the overview allows users to perform semi-targeted search for potentially interesting clusters.

Example 6 includes the subject matter of any of Examples 1-5, and further specifies that the overview incorporates the virtual bait compound within an isolation mode where a user can limit a depth of a displayed graph depth and release scoring restrictions without losing control over the graph.

Example 7 includes the subject matter of any of Examples 1-6, and further specifies that the virtual bait compound comprises in-silico data injected into the molecular networking algorithm.

Example 8 includes the subject matter of any of Examples 1-7, and further specifies that the fragmentation data of a reference compound is user-defined.

Example 9 includes the subject matter of any of Examples 1-8, and further includes a mass spectrometer, wherein the mass spectrometer is to generate data representative of the cluster of compounds within the sample.

Example 10 is a method for generating a candidate compound set based on mass spectrometry data, including: receiving, by a computing device, data representative of fragmentation of a reference compound; providing, by the computing device, the data to molecular networking algorithm as a virtual bait compound; generating, using the computing device, a cluster of compounds within a sample and surrounding the reference compound; and outputting, by the computing device, a candidate compound set based on the cluster of compounds.

Example 11 includes the subject matter of Example 10, and further specifies that the compounds are clustered by determining a similarity between the fragmentation data of the reference compound and real features detected in the sample.

Example 12 includes the subject matter of Example 11, and further specifies that determining the similarity between the fragmentation data and the real features is tuned based at least in part on the type of the virtual bait compound or an origin of the fragmentation data.

Example 13 includes the subject matter of any of Examples 10-12, and further specifies that a source of the fragmentation data of the reference compound comprises an artificially predicted spectrum based on known structure.

Example 14 includes the subject matter of any of Examples 10-12, and further specifies that a source of the fragmentation data of the reference compound comprises a cumulative spectrum based on diagnostic fragments of a class of similar compounds.

Example 15 includes the subject matter of any of Examples 10-14, further comprising:

allowing a user to perform a semi-targeted search among the candidate compound set.

Example 16 includes the subject matter of any of Examples 10-15, wherein a display including the candidate compound set incorporates the virtual bait compound within an isolation mode where a user can limit a depth of a displayed graph depth and release scoring restrictions without losing and further specifies that over the graph.

Example 17 includes the subject matter of any of Examples 10-16, and further specifies that the virtual bait compound comprises in-silico data injected into the molecular networking algorithm.

Example 18 includes the subject matter of any of Examples 10-17, and further specifies that the fragmentation data of a reference compound is user-defined.

Example 19 is a mass spectrometry system, comprising: a mass spectrometer; and a mass spectrometry support apparatus, wherein the mass spectrometry support apparatus is communicatively coupled to the mass spectrometer, and is configured to: provide fragmentation data of a reference compound to a molecular networking algorithm as a virtual bait compound; annotate the reference compound; generate, based on data about a sample generated by the mass spectrometer, a cluster of compounds within the sample and surrounding the annotated reference compound; and cause display of an overview of the cluster of compounds.

Example 20 includes the subject matter of Example 19, and further specifies that the compounds are clustered by determining a similarity between the fragmentation data of the reference compound and real features detected in the sample.

1. A mass spectrometry support apparatus, comprising:
first logic to receive fragmentation data of a reference compound;
second logic to provide the fragmentation data to a molecular networking algorithm as a virtual bait compound;
third logic to annotate the reference compound and generate a cluster of compounds within a sample and surrounding the annotated reference compound; and
fourth logic to provide for display an overview of structural relationships between the cluster of compounds.

2. The mass spectrometry support apparatus of claim 1, wherein the compounds are clustered by determining a similarity between the fragmentation data of the reference compound and real features detected in the sample.

3. The mass spectrometry support apparatus of claim 2, wherein determining the similarity between the fragmentation data and the real features is tuned depending on the type of the virtual bait compound or an origin of the fragmentation data.

4. The mass spectrometry support apparatus of claim 1, wherein a source of the fragmentation data of the reference compound comprises an artificially predicted spectrum based on known structure or a cumulative spectrum based on diagnostic fragments of a class of similar compounds.

5. The mass spectrometry support apparatus of claim 1, wherein the overview allows users to perform further semi-targeted search for clusters.

6. The mass spectrometry support apparatus of claim 1, wherein the overview incorporates the virtual bait compound within an isolation mode where a user can limit a depth of a displayed graph depth and release scoring restrictions without losing control over the graph.

7. The mass spectrometry support apparatus of claim 1, wherein the virtual bait compound comprises in-silico data injected into the molecular networking algorithm.

8. The mass spectrometry support apparatus of claim 1, wherein the fragmentation data of a reference compound is user-defined.

9. The mass spectrometry support apparatus of claim 1, further comprising:
a mass spectrometer, wherein the mass spectrometer is to generate data representative of the cluster of compounds within the sample.

10. A method for generating a candidate compound set based on mass spectrometry data, comprising:
receiving, by a computing device, data representative of fragmentation of a reference compound;
providing, by the computing device, the data to molecular networking algorithm as a virtual bait compound;
generating, using the computing device, a cluster of compounds within a sample and surrounding the reference compound; and
outputting, by the computing device, a candidate compound set based on the cluster of compounds.

11. The method of claim 10, wherein the compounds are clustered by determining a similarity between the fragmentation data of the reference compound and real features detected in the sample.

12. The method of claim 11, wherein determining the similarity between the fragmentation data and the real features is tuned based at least in part on the type of the virtual bait compound or an origin of the fragmentation data.

13. The method of claim 10, wherein a source of the fragmentation data of the reference compound comprises an artificially predicted spectrum based on known structure.

14. The method of claim 10, wherein a source of the fragmentation data of the reference compound comprises a cumulative spectrum based on diagnostic fragments of a class of similar compounds.

15. The method of claim 10, further comprising:
allowing a user to perform a semi-targeted search among the candidate compound set.

16. The method of claim 10, wherein a display including the candidate compound set incorporates the virtual bait compound within an isolation mode where a user can limit a depth of a displayed graph depth and release scoring restrictions without losing control over the graph.

17. The method of claim 10, wherein the virtual bait compound comprises in-silico data injected into the molecular networking algorithm.

18. The method of claim 10, wherein the fragmentation data of a reference compound is user-defined.

19. A mass spectrometry system, comprising:
a mass spectrometer; and
a mass spectrometry support apparatus, wherein the mass spectrometry support apparatus is communicatively coupled to the mass spectrometer, and is configured to:
provide fragmentation data of a reference compound to a molecular networking algorithm as a virtual bait compound;
annotate the reference compound;
generate, based on data about a sample generated by the mass spectrometer, a cluster of compounds within the sample and surrounding the annotated reference compound; and
cause display of an overview of the cluster of compounds.

20. The mass spectrometry system of claim 19, wherein the compounds are clustered by determining a similarity between the fragmentation data of the reference compound and real features detected in the sample.
